(19) United States
(12) Patent Application Publication
(10) Pub. No.: US 2006/0286108 A1
(43) Pub. Date: Dec. 21, 2006

(54) Topical compositions for the treatment of chronic wounds

(76) Inventor: Katherine A. Bell, Bellaire, TX (US)

Correspondence Address: LOCKE LIDDELL & SAPP LLP, 600 TRAVIS, 3400 CHASE TOWER, HOUSTON, TX, 77002-3095, US

(21) Appl. No.: 11/455,058

(22) Filed: Jun. 16, 2006

Related U.S. Application Data
(60) Provisional application No. 60/595,216, filed on Jun. 16, 2005.

(51) Int. Cl. A61K 39/395; A61K 31/63; A61K 31/16; A61K 31/655; A61K 31/137

(52) U.S. Cl. 424/145.1; 514/155; 514/156; 514/649; 514/629

(57) ABSTRACT

Methods for treating chronic wounds in a human are described by topically administering a dermatological composition comprising a TNF antagonist, a TACE inhibitor, a neutrophil antagonist, or a combination of a TNF antagonist and/or TACE inhibitor and a neutrophil antagonist. The TNF antagonist administered includes alefacept, efalizumab, etanercept, adalimumab, and onercept, while the neutrophil antagonist administered includes dapsone, colchicine, its analogs and prodrugs. The combination of TNF-antagonist and neutrophil antagonist administered includes sulfapyridine, sulfasalazine, mesalamine, and derivatives and prodrugs thereof. The topical compositions can be formulated to include the one or more of the antagonists in dissolved, semi-dissolved, and micro-particulate states.

### CROSS REFERENCE TO RELATED APPLICATIONS

The present application claims priority to U.S. Provisional Patent Application Ser. No. 60/595,216 filed Jun. 16, 2005, the contents of all of which are incorporated herein by reference.

### STATEMENT REGARDING FEDERALLY SPONSORED RESEARCH OR DEVELOPMENT

Not applicable.

### REFERENCE TO APPENDIX

Not applicable.

### BACKGROUND OF THE INVENTION

1. Field of the Invention

This disclosure relates generally to methods for the treatment of chronic wounds, and more particularly to methods for the treatment of chronic wounds by topical administration of dermatological compositions comprising tumor necrosis factor (TNF) antagonists, neutrophils (PMN) antagonists, and/or compounds which can act as both TNF antagonists and PMN antagonists.

2. Description of the Related Art

Wound repair is a highly orchestrated interplay involving several cell types, extracellular matrix components, and multiple soluble mediators, including growth factors and cytokines. Wound repair may be triggered by trauma, microbes or chemicals, which have caused tissue injury. For example, chronic leg and foot ulcers are often considered to be non-healing chronic wounds that occur as the result of a breakdown in the underlying physiology of the leg itself. Although restoration of tissue integrity is an innate host immune response, there are situations during which the wound repair process is impaired, such as in the instance of chronic wounds. Often, these breakdowns are associated with venous, arterial, or metabolic factors, or combinations of such factors. Consequently, such wounds are known to be a significant cause of morbidity, with a prevalence increasing as patients increase in age.

Clearly, there exists in the art a need to develop new methods and therapeutic agents to enhance chronic wound repair, particularly to treat skin wounds, surgical wounds, leg ulcers, diabetic ulcers, venous statsis, arthritic related ulcers, and the like.

Realization of the importance of this growing area of therapy has lead to an increasing number of developmental approaches to treating chronic wounds. Typically, the developments in therapy have been in the areas of physical methods and biological product-based methods. Physical methods have included topical negative pressure therapy at pressures in the range between −125 and −175 mmHg, therapeutic heat using specially designed warming blankets or mattreses (such as the Pegasus-Inditherm mattress), electrical stimulation [e.g., Gardner, S. E., et al., Wound Rep. Reg., Vol. 9: pp. 178-186 (2001)], vacuum therapy [Renner, R., et al., J. Dtsch. Dermatol. Ges., Vol. 4(6): pp. 468-475 (2006)], laser phototherapy, magnetism, cycloidal vibration therapy, and electromagnetic therapy, such as applied in the treatment of pressure ulcers.

Biological therapies which have been applied to chronic wounds range from the use of larva therapy (also termed maggot therapy and biosurgery) using the larvae of the green bottle fly Lucilia Sericata, to skin substitutes such as the AlloDerm® (LifeCell Corporation) and TransCyte® (Smith & Nephew) bioengineered skin layer products based on tissue engineering advances. Other areas under investigation, include the applications of stem cell therapy, growth factors such as platelet derived growth factors (PDGF), such as REGRANEX® gel (Johnson & Johnson), keratinocyte growth factor 2 (KGF 2), and gene therapy for the treatment of chronic wounds, such as diabetic foot ulcers. Other biological approaches have included the use of fibrin sealant in order to accelerate revascularization and decrease wound contraction, and platelet “gels” prepared from autologous blood and which allegedly provide growth factors and proteins like fibronectin.

Not all approaches have been physical or biological in nature. Advances in technology have made multidisciplinary and varied approaches more acceptable. Notable among such “miscellaneous” therapies being investigated is the use of hyperbaric oxygen in the treatment of wounds [Wang, C., et al., Arch. Surg., Vol. 138: pp. 272-279 (2003)], based on the idea of increasing oxygen delivery to the tissues in the wound, so as to enhance tissue oxygenation and consequently tissue regeneration. Other approaches include the area of hybrid or composite dressings, including films, hydrocolloids, alginates derived from seaweed, and protease modulating matrix dressings, such as PROMOGRAN® Matrix Wound Dressing (Johnson & Johnson).

Regardless of these advances in the treatment of chronic wounds, the growing incidence of these types of wounds requires further treatment options to be developed. This application for patent discloses methods for treating chronic wounds in patients using dermatological formulations.

### BRIEF SUMMARY OF THE INVENTION

In a first aspect of the present invention, methods of healing chronic wounds in patients in need of such healing are described, wherein the method comprises the step of topically administering to the patient a dermatological composition comprising a therapeutically effective amount of one or more TNF antagonists, TACE inhibitors, or combinations thereof, a neutrophil antagonist, a combination TNF antagonist/TACE inhibitors/neutrophil antagonists, or a combination thereof. In association with this aspect of the invention, the dermatological composition can further comprise one or more additional therapeutic agents, including but not limited to antibacterial agents, antifungal agents, antimycobacterial agents, nonsteroidal anti-inflammatory drugs, and combinations of such therapeutic agents. In further accordance with this aspect of the present invention, the dermatological formulation can be a cream, an ointment, a gel, a hydrogel, a topical aerosol, or a subcutaneous formulation suitable for application topically.

In a further aspect of the present invention, a method for inhibiting the action of one or more tumor necrosis factors (TNF) or TNF-α-converting enzymes (TACEs) for the purpose of treating chronic wounds in a human patient is described, wherein the method comprises topically administering a dermatological composition comprising a therapeutically-effective dosage level of a TNF antagonist selected from the group consisting of adalimumab, alefacept, efalizumab, etanercept, and onercept, as well as mixtures thereof.

In another aspect of the present invention, a method for inhibiting the action of neutrophils for treating chronic wounds in a patient is described. This method comprises topically administering a dermatological composition comprising a therapeutically-effective dosage level of a neutrophil antagonist, wherein the neutrophils antagonist is selected from the group consisting of dapsone, colchicine, colchicine analogs, colchicine prodrugs, and colchicine polymorphs.

In a further aspect of the present invention, a method for inhibiting the action of one or more tumor necrosis factors (TNF), TNF-α-converting enzymes (TACEs), neutrophils, or any combination thereof (e.g., TNF and neutrophils simultaneously) for treating chronic wounds in patients is described. This method comprises topically administering a dermatological composition comprising a therapeutically-effective dosage level of at least one tumor necrosis factor antagonist and at least one neutrophil antagonist. In accordance with this aspect of the invention, the at least one tumor necrosis factor antagonist and at least one neutrophil antagonist can be the same compound or therapeutically active agent, and as such can be selected from the group of compounds exhibiting both anti-neutrophilic and anti-TNF behavior comprising sulfasalazine, mesalamine, sulfapyridine, and combinations thereof.

In another aspect of the present invention, a wound dressing having a layered structure for the controlled release of active substance to chronic wounds is described, wherein the dressing comprises a hydrocolloid-containing swellable hydrogel and at least one active substance in at least one of the layers. In accordance with this aspect of the invention, the at least one active substance is a biologically active peptide or protein, a TNF antagonist, a TNF-alpha antagonist, a neutrophil antagonist, or a combination thereof, wherein the biologically active peptide or protein may or may not be equivalent to the TNF antagonist, TNF-alpha antagonist, or neutrophil antagonist, and is preferably selected from the group comprising adalimumab, alefacept, efalizumab, etanercept, onercept, or a combination of two or more biologically active peptides or proteins.

In a further aspect of the present invention, a method of healing a chronic wound in a patient in need thereof, the method comprising the step of subcutaneously administering to the patient a dermatological composition comprising a therapeutically effective amount of a TNF antagonist, a neutrophil antagonist, a combination TNF antagonist/neutrophil antagonist, or a combination thereof in a therapeutically effective amount. In accordance with this aspect of the present invention, the chronic wound may be selected from the group consisting of venous stasis ulcers, diabetic ulcers, pressure ulcers, vasculitis-induced ulcers, arterial ulcers, and pyoderma gangrenosum. In further accordance with this aspect, the subcutaneous dermatological composition is a hydrogel having a viscosity from about 1 mPa*s to about 2500 mPa*s.

### DEFINITIONS

The following definitions are provided in order to aid those skilled in the art in understanding the detailed description of the present invention.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

Since prodrugs are known to enhance numerous desirable qualities of pharmaceuticals (e.g., solubility, bioavailability, manufacturing, etc . . . ) the compounds of the present invention may be delivered in prodrug form. Thus, the present invention is intended to cover prodrugs of the presently claimed compounds, methods of delivering the same and compositions containing the same. “Prodrugs” are intended to include any covalently bonded carriers which release an active parent drug of the present invention in vivo when such prodrug is administered to a mammalian subject. Prodrugs of the present invention are prepared by modifying functional groups present in the compound in such a way that the modifications are cleaved, either in routine manipulation or in vivo, to the parent compound. Prodrugs include compounds of the present invention wherein a hydroxy, amino, or sulfhydryl group is bonded to any group that, when the prodrug of the present invention is administered to a mammalian subject, it cleaves to form a free hydroxyl, free amino, or free sulfhydryl group, respectively. Examples of prodrugs include, but are not limited to, acetate, formate and benzoate derivatives of alcohol and amine functional groups in the compounds of the present invention.

“Stable compound” and “stable structure” are meant to indicate a compound that is sufficiently robust to survive isolation to a useful degree of purity from a reaction mixture, and formulation into an efficacious therapeutic agent.

The term “topical” as used herein refers to the route of administration of a dermatological composition that involves direct application to the body part being treated, e.g., the skin. Examples of topical application include application to the skin of gels, creams, ointments, or other semisolids to rub-on, solutions to spray, or liquids to be applied by an applicator or by a drip/flush method. Rinse-off application with washes, cleansers, or shampoos are also examples of topical application. Typically, areas of the body suitable for application of the dermatological compositions described herein include but are not limited to the skin of the legs, arms, feet, chest, back, buttocks, and other skin sites where chronic wounds, such as ulcers and ulcerous lesions, may occur, as well as dermal mucous membranes, such as the mouth, anal areas, and genital areas.

As used herein, “treating” or “treatment” refer to the treatment of a disease-state in a mammal, particularly in a human patient, and include: (a) preventing the disease-state from occurring in a mammal, in particular, when such mammal is predisposed to the disease-state but has not yet been diagnosed as having it; (b) inhibiting the disease-state, i.e., arresting it development; and/or (c) relieving the disease-state, i.e., causing regression of the disease state.

“Therapeutically effective amount” is intended to include an amount of an agent of the present invention or an amount of the combination of agents described herein claimed effective to inhibit TNF (such as TNF-alpha), TNF-α-converting enzyme (TACE), neutrophils, or a combination of TNF, TACE, and neutrophils in a patient having one or more chronic wounds. In some embodiments, such combinations of compounds can preferably be or form a synergistic combination. Synergy, as used herein, is intended to refer to that effect as described for example by Chou and Talalay [Adv. Enzyme Regul., 22:27-55 (1984)], wherein synergy occurs when the effect (in this case, inhibition of the desired target) of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased chronic-wound healing effect, or some other beneficial effect of the combination compared with the individual components.

### DETAILED DESCRIPTION

One or more illustrative embodiments incorporating the invention disclosed herein are presented below. Not all features of an actual implementation are described or shown in this application for the sake of clarity. It is understood that in the development of an actual embodiment incorporating the present invention, numerous implementation-specific decisions must be made to achieve the developer's goals, such as compliance with system-related, business-related, government-related and other constraints, which vary by implementation and from time to time. While a developer's efforts might be complex and time-consuming, such efforts would be, nevertheless, a routine undertaking for those of ordinary skill the art having benefit of this disclosure.

In general terms, Applicant has created a method of topically treating chronic wounds, especially chronic skin and soft tissue wounds including diabetic ulcers, decubitis ulcers, pressure-related ulcers, venous stasis ulcers, vasculitis-induced ulcers, arterial ulcers, pyoderma gangrenosum, and arthritis-related ulcers, in patients diagnosed with such diseases. In accordance with the present invention, TNF-alpha within the wounds may be inhibited, TACE within the wounds may be inhibited, neutrophil levels within the wounds may be inhibited, and/or one or more of neutrophil, TACE and TNF-alpha levels within the wound may be lowered, allowing such chronic wounds to heal. Combinations of drugs comprising the compounds of the present disclosure in topical treatment regimens for chronic wound diseases are also contemplated herein.

The present invention envisions methods for the treatment of chronic wounds, including diabetic ulcers, decubitis ulcers, pressure-related ulcers, venous stasis ulcers, vasculitis-induced ulcers, arterial ulcers, pyoderma gangrenosum, and arthritis-related ulcers. The method for treatment of a patient currently afflicted with one or more chronic wounds, in accordance with the present disclosure, comprises topically administering to the patient in need of such treatment a therapeutically effective amount of one or more compounds selected from tumor necrosis factor (TNF) antagonists, TACE-inhibitors, neutrophil (polymorphonuclear neutrophil; PMN) antagonists, and compounds which can act as both TNF-antagonists and PMN-antagonists, or TACE-inhibitors and PMN-antagonists.

Therapeutic Agents

In accordance with embodiments and aspects of the present invention, the dermatological compositions to be used for the treating of chronic wounds can comprise TNF antagonists (anti-TNF/TNF-alpha compounds), including TACE inhibitors or antagonists, and/or neutrophil (PMN) antagonists (anti-neutrophilic compounds).

TNF antagonists, and more particularly anti-TNF-alpha agents as described herein refer to those compounds or biological agents (e.g., biologically active peptides or proteins) which inhibit the mechanism of action of TNF and/or TNF-alpha, either directly or indirectly. Suitable TNF antagonists and/or anti-TNF-alpha compounds for use in the methods and compositions of the present invention include but are not limited to alefacept (AMEVIVE™; Astellas Pharma US, Inc., Deerfield, Ill.); Efalizumab (RAPTIVA®; Genentech, Inc., CA); etanercept (ENBREL®; Immunex Corporation, CA); adalimumab (HUMIRA®; Abbott Laboratories, IL); onercept, a recombinant TNF binding protein (r-TBP-1) (Serono International S.A., Switzerland); D2E7, a human anti-TNF monoclonal antibody (Knoll Pharmaceuticals, Abbott Laboratories); CDP 571 (a humanized anti-TNF IgG4 antibody); CDP 870 (an anti-TNF alpha humanized monoclonal antibody fragment), both from Celltech; soluble TNF receptor Type I (Amgen); and pegylated soluble TNF receptor Type I (PEGs TNF-R1) (Amgen), as well as variants, fragments, and recombinant proteins that are at least 70% similar to these peptides and proteins, and combinations of such TNF antagonists and/or anti-TNF-agents. All such biological agents can be obtained using known synthetic methodologies, including solid phase synthesis, solution phase synthesis methods, and biological synthesis methods known to those of skill in the art, using for example, vectors and recombinant technologies.

Biologics such as those listed above have been developed which have been shown to offer dramatic clinical benefit for systemic illnesses in humans, even for those disorders which have not responded to large and repeated doses of corticosteroids. These biologics fall into the category of cytokine antagonists because they block, or antagonize, the biologic action of a specific cytokine which has adverse clinical effects. These cytokines include the pro-inflammatory cytokines in the TNF family, as well as the recently elucidated TACE (TNF-alpha converting enzyme) family of enzymes [see, Armstrong, L, et al., Am. J. Respir. Cell Mol. Biol., Vol. 34(2): pp. 219-225 (2006), and references cited therein]. Thus, in accordance with aspects of the present invention, therapeutic agents that can be classified as TNF-antagonists for use in preparing the dermatological formulations for the treatment of chronic wounds can also include TACE inhibitors, including but not limited to sultam hydroxamates and pipecolic acid-based compounds, as well as biological variants of adalimumab, alefacept, efalizumab, etanercept, and onercept. For the purposes of the present disclosure, “antagonist”, “inhibitor”, and “blocker” are used interchangeably.

Recent research has demonstrated that new TNF and/or neutrophil antagonists can be manufactured from an existing molecule by subtracting a portion of the amino acid sequence from the molecule. This has the advantage of making the molecule smaller. This smaller molecule can be easier to manufacture and may have clinical advantages, such as reduced immunogenicity in the human in vivo. Therefore, the biologically active molecules of consideration herein shall also include, in addition to those specified, any molecule which contains a fragment of any of the named molecules (e.g., etanercept, efalizumab, or alefacept), such molecules including recombinant variants of the biologically active molecule. A fragment as used herein shall be defined as an identical amino acid sequence 50% or greater in length of the original molecule and possessing TNF binding capability.

Anti-neutrophilic (PMN) compounds (neutrophil antagonists) as described herein refers to those compounds capable of inhibiting the mechanism of action of neutrophils directly or indirectly. Compounds suitable for use herein as neutrophil antagonists include dapsone [4-(4-aminophenyl)sulfonyl aniline, (Atrisone®); Atrix Laboratories, Inc.], colchicine, or combinations thereof. As used herein, dapsone is meant to include both 4-(4-aminophenyl)sulfonyl aniline (also known as 4,4′-diaminodiphenyl sulfone, diaphenylsulfone, and 4,4′-sulfonyldianiline), such as available commercially or by synthetic methods known in the art, as well as dapsone analogs and derivatives as described in U.S. Pat. No. 4,829,058 and U.S. Pat. No. 4,912,112, as well as those dialkyldithiocarbamates described by Makarov, et al., [J. Antimicrob. Chemother., Vol. 57, pp. 1134-1138 (2006)], dapsone hydrates, dapsone solvates, dapsone polymorphs, and dapsone prodrugs. Colchicine, as used herein, is meant to include the compound colchicine as obtained from known synthetic methods [e.g., Nakamura, et al., Chem. Pharm. Bull., Vol. 10, pp. 281-304 (1962); Evans, D. A., et al., J. Am. Chem. Soc., Vol. 103, pp. 5813-5821 (1981)] or via isolation methods from the autumn crocus, Colchicum automnale L., as well as colchicine analogs and derivatives, including but not limited to demecolcine (colcemid), ±colchicine, ±isocolchicine, and ±desacetamidoisocolchicine, colchicine hydrates, colchicine solvates, colchicine prodrugs, and colchicine polymorphs, alone or in combination with other neutrophil antagonists.

Compounds suitable for use with the methods of the present invention which can be classified as exhibiting both anti-neutrophilic and anti-TNF-alpha behavior, and are thereby compounds which are both neutrophil antagonists and TNF-alpha antagonists, include sulfasalazine, mesalamine, and combinations of both sulfasalazine and mesalamine. As used herein, sulfasalazine is meant to include sulfasalazine and sulfapyridine, as well as analogs and derivatives of sulfasalazine, sulfasalazine prodrugs, sulfasalazine hydrates, sulfasalazine solvates, and sulfasalazine polymorphs as suitable compounds exhibiting both anti-neutrophilic and anti-TNF-alpha behavior. Similarly, as used herein, mesalamine is meant to include mesalamine (N-acetyl-5-amino salicylic acid; 5-ASA), as well as analogs and derivatives of mesalamine, mesalamine prodrugs such as olsalazine (DIPENTUM®) and balsalizide (COLAZAL®), mesalamine hydrates, mesalamine solvates, and mesalamine polymorphs, and mixtures thereof, as suitable compounds exhibiting both anti-neutrophilic and anti-TNF-alpha behavior.

Additional Therapeutic Agents

In accordance with aspects of the present invention, the topical formulations described herein comprising one or more of the primary therapeutic agents described above can further comprise one or more additional therapeutic agents, as desired, for administration concurrently with the primary therapeutic agents. Such additional therapeutic agents can include, but are not limited to, antibacterial therapeutic agents, antifungal therapeutic agents, antimycobacterial therapeutic agents, non-steroidal anti-inflammatory drugs (NSAIDS), and combinations thereof (e.g., an antibacterial therapeutic agent and an antifungal therapeutic agent).

Antibacterial therapeutic agents suitable for use with the topical formulations of the present invention include, but are not limited to, those antibacterial agents effective against, gram-positive, gram-negative, or both gram-positive and gram-negative bacteria. Suitable antibacterial agents for use in formulations of the present invention include beta-lactams, such as penicillins (e.g., penicillin G, oxacillin, ampicillin, nafcillin, ticarcillin, and amoxicillin), cephalosporins (e.g., cephalothin, cephalexin, cefazolin, cephradine, cephapirin, cefamandole, cefoxitin, cefoperazone, cefotaxime, and ceftriaxone), monbactams (e.g., aztreonam), beta-lactamase inhibitors (e.g., clavulanic acid, sulbactam, and tazobactam), and carbapenems (e.g., imipenem), as well as derivatives of such beta-lactams; polypeptides, such as bacitracin; aminoglycosides, such as neomycin, gentamicin, clindamycin, tobramycin, amikacin, netilmicin, and lincomycin; amino-glycosidic-type compounds, such as spectinomycin; macrolides, such as erythromycin, streptomycin, azithromycin, and clarithromycin; azoles such as metronidazole (FLAGYL®); mupirocin (BACTROBAN®); azines, such as silver sulfadiazine; inhibitors of bacterial cell wall synthesis, such as vancomycin, teicoplanin, and fosfomycin; and combinations of two or more antibacterial agents.

The antifungal therapeutic agents suitable for use with the formulations of the present invention in the treatment of chronic wounds include, but are not limited to, azoles, such as miconazole, clotrimazole, ketoconazole, oxiconizole, econazole, sulconazole, fluconazole, and itraconazole; allylamines, such as naftifine (NAFTIN®) and terbinafine (LAMISIL®); benzylamines, such as butenafine; polyenes, such as nystatin and amphotericin B; thiocarbonates, such as tolnaftate; sulfides, such as selenium sulfide; nitrogen-containing heterocycles, such as ciclopirox (LOPROX®), and combinations of two or more antifungal agents.

Antimycobacterial therapeutic agents suitable for use with the formulations of the present invention include but are not limited to, rifampin, clofazimine, sulfones, ciprofloxacin, ofloxacin, cycloserine, capreomycin, ethionamide, pyrazinamide (PZA), ethambutol, and combinations of two or more antimicrobial therapeutic agents.

Non-steroidal anti-inflammatory drugs suitable for use in combination with the formulations of the present invention include, but are not limited to, aspirin, ibuprofen (e.g., MOTRIN®), ketoprofen (Orudis), salsalate (Amigesic), diflunisal (Dolobid), nabumetone (Relafen), Piroxicam (Feldene), naproxen (e.g., Aleve, Naprosyn), diclofenac (voltaren), indomethacin (Indocin), sulindac (Clinoril), tolmetin (Tolectin), etodolac (Lodine), ketorolac (Toradol), oxaprozin (Daypro), and COX-1 and/or COX-2 inhibitors, including celecoxib (CELEBREX®), etoricoxib (ARCOXIA®), and rofecoxib (VIOXX®), as well as combinations of two or more NSAIDS.

Dosage and Formulation

The compounds of the present invention can be used in the form of pharmaceutically acceptable salts derived from inorganic or organic acids. As elucidated above, by “pharmaceutically acceptable salt” it is meant those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zürich, Switzerland: 2002). The salts can be prepared in situ during the final isolation and purification of the compounds of the present invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Preferred salts of the compounds of the present invention include but are not limited to phosphate, tris and acetate.

Pharmaceutically acceptable salts may be also obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids can also be made.

The formulations may conveniently be presented in unit dosage form and may be prepared by any of the methods well known in the art of pharmacy. All methods include the step of bringing into association a compound of the invention or a pharmaceutically acceptable salt or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired topical formulation.

The compound or a pharmaceutically acceptable ester, salt, solvate, polymorph or prodrug as appropriate can be mixed with other active materials that do not impair the desired action, or with materials that supplement the desired action, including other drugs against chronic wound disorders. Solutions or suspensions used for topical application can include, for example, the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The topical preparation can be enclosed in ampoules, disposable tubes, or multiple dose vials made of glass or plastic. Optionally, and equally acceptable, the topical preparations can be in the form of transdermal patches or wraps.

The compounds of this invention can be administered by any means that produces contact of the active agent with the agent's site of action in the body of a mammal. The active ingredient(s) can be administered alone, but is generally administered with one or more pharmaceutical carriers. They can be administered by any conventional means available for use in conjunction with pharmaceuticals, either as individual therapeutic agents or in a combination of therapeutic agents, e.g., with one or more anti-bacterial agents, anti-fungal agents, etc., as mentioned above. They can be administered alone, but generally are administered with a pharmaceutical carrier selected on the basis of the chosen route of administration (topical) and standard pharmaceutical practice. A valuable treatise with respect to pharmaceutical dosage forms is Remington's Pharmaceutical Sciences, Mack Publishing, incorporated herein by reference.

The compounds of the present invention can be administered in such topical dosage forms as topical gels, creams, lotions, solutions, or suspensions. Likewise, they may also be administered using a pharmaceutical or cosmetic carrier form, such as an ointment, roll-on or stick product, micro-emulsion, shake powder, an aerosolized spray or mousse, a pump spray or mousse, or a bath additive.

Examples of ointments include but are not limited to essentially non-aqueous mixtures of petrolatum, lanolin, polyethylene glycol, plant or animal oils, either hydrogenated or otherwise chemically modified. An ointment may also contain a solvent in which a compound (e.g., etanercept, colchicine, or dapsone) is either fully or partially dissolved. Additional pharmaceutical carriers, as described herein, will be known to those skilled in the art and this list is not to be considered to be limiting.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. An ordinarily skilled physician or veterinarian can readily determine and prescribe the effective amount of the drug required to prevent, counter, or arrest the progress of the condition

By way of general guidance, the daily topical dosage of each active ingredient (e.g., TNF antagonist, TACE antagonist, neutrophil antagonist, or combination TNF/neutrophil antagonist as described herein), when used for the indicated effects, will range between about 0.001 to 1000 mg/kg of body weight per day, preferably between about 0.01 to about 100 mg/kg of body weight per day, and more preferably between about 1.0 to about 50 mg/kg/day. For a normal male adult human of approximately 70 kg of body weight, this translates into a dosage of about 70 to about 3500 mg/day. As a topical liquid application, such as by topical liquid drip or flushing of the liquid formulation over the top of the chronic wound, the most preferred doses will range from about 0.01 to about 100 mg/kg/minute during a constant rate application. Advantageously, compounds of the present invention may be administered in a single daily dose, or the total daily dosage may be administered in divided doses of two, three, four, five, six, seven, eight, nine, or ten times daily.

In the methods of the present invention, the compounds herein described in detail can form the active ingredient, and are typically administered in admixture with suitable pharmaceutical diluents, excipients, or carriers (collectively referred to herein as carrier materials) suitably selected with respect to the intended form of administration, that is, topical creams, ointments, gels, hydrogels, and the like, and consistent with conventional pharmaceutical practices.

The compounds of the present invention can also be administered in the form of topical liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Compounds of the present invention may also be coupled with soluble polymers as targetable drug carriers, for use in topical administration. Such polymers can include polyvinylpyrrolidone, pyran copolymer, polyhydroxypropylmethacrylamide-phenol, polyhydroxyethyl-aspartamidephenol, or polyethyleneoxide-polylysine substituted with palmitoyl residues. Furthermore, the compounds suitable for use with the methods of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

In accordance with certain aspects of the present invention, dosage forms (pharmaceutical compositions) suitable for administration topically may contain from about 1 mg to about 500 mg of active ingredient per dosage unit. In these pharmaceutical compositions the active ingredient will ordinarily be present in an amount of about 0.5-95% by weight based on the total weight of the composition.

Suitable pharmaceutical carriers for topical, dermatological formulations are described in Remington's Pharmaceutical Sciences, Mack Publishing Company, a standard reference text in this field.

As indicated above, the primary therapeutic agents of the present invention may be administered in combination with one or more additional, or secondary, therapeutic agents, including but not limited to anti-bacterial agents, antimycobacterial agents, anti-fungal agents, and non-steroidal anti-inflammatory drugs (NSAID's). The agents of the present invention and such second therapeutic agent can be administered separately or as a physical combination in a single dosage unit, in any dosage form and by various routes of administration, as described above.

The compounds of the present invention may be formulated together with the second therapeutic agent in a single dosage unit (that is, combined together in one gel, ointment, cream, aerosol, etc.). When the first therapeutic agent and the second therapeutic agent are not formulated together in a single dosage unit, the first therapeutic agent and the second therapeutic agent may be administered essentially at the same time, or in any order; for example the first therapeutic agent may be administered first, followed by administration of the second agent. When not administered at the same time, preferably the administration of the first therapeutic agent and the second therapeutic agent occurs less than about one hour apart, more preferably less than about 5 to 60 min apart.

The dosage of the first therapeutic agent when administered alone or in combination with a second therapeutic agent may vary depending upon various factors such as the pharmacodynamic characteristics of the particular agent and its mode and route of administration, the age, health and weight of the recipient, the nature and extent of the symptoms, the kind of concurrent treatment, the frequency of treatment, and the effect desired, as described above.

Particularly when provided as a single dosage unit, the potential exists for a chemical interaction between the combined active ingredients. For this reason, when the first therapeutic agent and a second therapeutic agent are combined in a single dosage unit they are formulated such that although the active ingredients are combined in a single dosage unit, the physical contact between the active ingredients is minimized (that is, reduced). For example, one active ingredient may be enteric coated. By enteric coating one of the active ingredients, it is possible not only to minimize the contact between the combined active ingredients, but also, it is possible to control the release of one of these components in the intestinal tract, such as may be the case when a formulation of the present invention is applied to a dermal mucus membrane area. One or more of the active ingredients (e.g., primary therapeutic agents) may also be coated with a sustained-release material which effects a sustained-release throughout the dermis, epidermis, and upper layers of the skin, and also serves to minimize physical contact between the combined active ingredients. Furthermore, the sustained-released component can be additionally enteric coated such that the release of this component occurs only in the desired physiological region. Still another approach would involve the formulation of a combination product in which the one component is coated with a sustained and/or enteric release polymer, and the other component is also coated with a polymer such as a low-viscosity grade of hydroxypropyl methylcellulose (HPMC) or other appropriate materials as known in the art, in order to further separate the active components. The polymer coating serves to form an additional barrier to interaction with the other component.

These as well as other ways of minimizing contact between the components of combination products of the present invention, whether administered in a single dosage form or administered in separate forms but at the same time by the same manner, will be readily apparent to those skilled in the art, once armed with the present disclosure.

Exemplary Topical Formulations

In accordance with aspects of the present disclosure, the therapeutic agents of the present invention can be formulated and applied as a variety of topical formulations, including but not limited to creams, lotions, topical solutions or suspensions, and aerosol sprays. In one aspect of the present invention, the therapeutic agents may be applied as a topical cream or lotion in which one or more therapeutic agents selected from the group consisting of TNF antagonists, TNF-alpha antagonists, TACE-inhibitors, neutrophil inhibitors, combination TNF/neutrophil antagonists, and combinations thereof, are dissolved, dispersed, or suspended, either fully or partially (e.g., either fully or partially dissolved, or either fully or partially dispersed). Topical creams or lotions may be oil-in-water emulsions, water-in-oil emulsions, or other emulsions known to those of skill in the art. Oil phases, if present, may include, but are not limited to, fatty alcohols, fatty acids, fatty esters, such as cetyl palmitate, cetyl alcohol, stearyl alcohol, stearic acid, isopropyl stearate, glycerol stearate, mineral oil, white petrolatum, or other oils, alone or in combination.

In another aspect of the present invention, one or more of the therapeutic agents as described herein (e.g., one or more therapeutic agents selected from the group consisting of TNF antagonists, TNF-alpha antagonists, TACE-inhibitors, neutrophil inhibitors, combination TNF/neutrophil antagonists, and combinations thereof), may be applied as a solution or suspension. These are fluid solvent or mixed-solvent systems including, but not limited to, water, ethanol, isopropanol, propylene glycol, glycerol, polyethylene glycol, ethyl acetate, propylene carbonate, n-methyl pyrolidone, triethanolamine, 1,4-butanediol, triacetin, diacetin, dimethyl isosorbide, alone or in combination.

All of the above-described topical formulations, topical creams or lotions and topical solutions or suspension, can also further comprise a number of optional ingredients, including but not limited to preservatives, antioxidants, fragrances, colorants, sunscreens, thickeners, emulsifiers, suspending agents, enhancers, and other additives necessary to achieve pharmaceutically or cosmetically acceptable or preferred product formulations. Such suitable additives are known in the art and are described, for example, in Kibbe, A. H., “Handbook of Pharmaceutical Excipients”, 3^{rd} Ed., Pharmaceutical Press (2000). For example, emulsifiers that can be added to topical formulations of the present invention include steareth 20, ceteth 20, sorbitan sesquioleate, sorbitan mono-oleate, propylene glycol stearate, sodium lauroyl sarcosinate, polysorbate 60, monodiglycerides, esters (e.g., lactic or citric esters) of monodiglycerides, polyglycerol esters, or combinations thereof. Similarly, preservatives (ingredients that prevent or retard microbial (both gram positive and gram negative), algeal, or fungal growth and protect formulations from spoilage) that can be used in forming the topical, dermatological formulations of the present invention can include from about 0.05 wt. % to about 5 wt. % by weight of the total composition, suitable preservatives including parabens (alkyl esters of p-hydroxy benzoic acid), such as methylparaben, propylparaben, butylparaben, and the like; chloroxylenol; benzoic acid; sodium benzoate; benzyl alcohol; phenoxyethanol; chlorocresol; DMDM Hydantoin; 3-iodo-2-propylbutyl carbamate; potassium sorbate; E200 sorbic acid; methyl salicylate; chlorhexidine digluconate; hexamine hippurate; cetylpyridinium chloride; quaternary ammonium compounds, such as benzylalkonium chloride and cetrimide (CTAB; alkyltrimethyl ammonium bromide); dequalinium; Triclosan, or combinations thereof.

It should be noted that none of the topical formulations as recited or described herein are meant to be limited in any way to these components, as one skilled in the art of formulations will be aware of additional components useful in the formulation of topical creams, lotions, gels, suspensions, solutions, aerosols, ointments, bath additives, medicinal bandages, transdermal patches, and the like.

Hydrogel Formulations

In accordance with certain aspects of the present invention, the therapeutic agents as described herein can be formulated for topical (and/or subcutaneous) administration by way of being prepared as a hydrogel formulation. As used herein, the term “hydrogel” is meant to refer to those three-dimensional, hydrophilic, polymeric networks capable of imbibing large amounts of water or biological fluids and composed of hompolymers and/or copolymers, having a reduced solubility due to the presence of chemical cross-links (e.g., tie-points or junctions) or physical cross-links (e.g., entanglements or crystallites), and exhibiting a thermodynamic compatibility with water that allows them to swell in aqueous media [see, Peppas, N. A., et al., in “Hydrogels in Medicine and Pharmacy, Vol. 1”; Peppas, N. A., Ed.; CRC Press, Boca Raton, Fla.: pp. 1-27 (1986)], including all those suitable hydrogels described by Peppas, et al. in European Journal of Pharmaceutics and Biopharmaceutics [Vol. 50; pp. 27-46 (2000)], which is incorporated herein in its entirety. Such hydrogels suitable for use herein include both neutral and ionic hydrogels, based on the nature of their side groups, as well as those classified as affine or phantom networks, preferably physiologically-responsive hydrogels [Peppas, N. A., J. Bioact. Compat. Polym., Vol. 6; pp. 241-246 (1991)], and includes pH-sensitive hydrogels, temperature-sensitive hydrogels, and other stimuli-sensitive hydogels, including light-, magnetic field-, electric current-, and ultrasound-sensitive hydrogels.

In accordance with aspects of the present invention, the topical (and subcutaneous) formulations described herein can include one or more diffusion-inhibiting substances, which may act or contribute to the act of inhibiting the diffusion of the therapeutically active agents with tissue in the wound when the formulation is administered. As used herein, diffusion-inhibiting substances refer to those gel-forming and hydrogel-forming substance and other, suitable non-particulate matrices, including both natural and synthetic polymeric materials.

In accordance with these aspects of the invention, any conventional pharmaceutically or physiologically acceptable polymer and gel forming agents may be utilized to produce the hydrogels and aqueous gels of the invention. These gel forming agents can act inhibit the diffusion of the active ingredient with human tissue when the composition is administered to a human patient topically, or subcutaneously. These conventional polymeric gel forming agents include cellulose derivatives, alginic acid derivatives, dextrans, hyaluronic acids, dermatan and heparan sulphates, gelatins, collagen, polyvinylpyrrolidone, polyvinyl alcohol, poly(meth)acrylic acids, poly(meth)acrylates and/or their derivatives. Derivatives are chemically modified entities obtained by amidation, alkylation, carboxymethylation, addition of fatty acid units or addition of polyethyleneglycol-units (so called ‘pegylation’). To get the necessary gel forming properties the polymers additionally may be cross-linked by e.g. glutardialdehyde. In case of acidic compounds (alginic acid, polymethacrylates) derivatives are also their salts (such as ammonium-, barium-, magnesium-, calcium-salt). These compounds are known in the art and described for example in Ash, M. and I.: Handbook of Pharmaceutical Additives. Gower, 1995, 887f.; 915-918; Fiedler, H. P.: Lexikon der Hilfsstoffe. ecv, 1998; Kibbe, A. H.: Handbook of Pharmaceutical Excipients. 3rd ed., Pharmaceutical Press, 2000. Preferably, the natural polymer is selected from the group consisting of cellulose derivatives, alginic acid derivatives, dextrans, gelatines, collagen, hyaluronic acids and/or dermatan and heparan sulphates. The cellulose derivatives are preferred for use as diffusion-inhibiting substances in the meaning of certain aspects of the present invention. In the case of the cellulose derivatives, those preferred include, but are not limited to, the soluble alkyl- or hydroxyalkylcellulose derivatives, such as hydroxymethylcellulose (HMC), hydroxyethylcellulose (HEC), hydroxypropylcellulose (HPC), methylhydroxyethylcellulose (MHEC), hydroxypropylmethylcellulose (HPMC), carboxymethylcelluose (CMC), methylcellulose (MC) or their mixtures.

Subcutaneous Formulations

In accordance with aspects of the present invention, the therapeutic agents described herein can also be formulated for subcutaneous, parenteral administration directly to a chronic wound, as well as the preparation of such formulations. By means of such subcutaneous administration locally of the therapeutic agents described herein (e.g., TNF antagonists, TACE inhibitors, neutrophil antagonists, and the like), relatively high, therapeutically relevant concentrations of the therapeutic agents (e.g., TNF antagonists or neutrophil antagonists) can be obtained almost immediately within the chronic wound, without the occurrence of incompatibilities. Such preparations in turn can allow for medically significant treatment regimes with administrations at long time intervals (e.g., ≧4 weeks) to be possible.

In accordance with one preferred embodiment of the present invention, gel-like, aqueous pharmaceutical preparations for subcutaneous administration containing one or more therapeutically active agents as described herein, such as TNF antagonists, TNF-alpha antagonists, TACE inhibitors, neutrophil antagonists, combination TNF/neutrophil antagonists, and the like. Such pharmaceutical preparations may be characterized in that the pharmaceutical preparation contains at least one compound that inhibits the diffusion of the active substance(s) in the tissue of, in and around a chronic wound, such as a diabetic ulcer or bedsore. In further accordance with this aspect of the present invention, the therapeutically active agents may be present, in dissolved or dispersed form, in the preparation.

In further accordance with this aspect of the present invention, the preparations produced for use as subcutaneous preparations, typically in the form of a gel, hydrogel, liquid, or the like, may have a viscosity in an appropriate range (in a unit dosage form suitable for subcutaneous administration) such that the subcutaneous preparations are capable of being administered by means of suitable injection needles. In accordance with this aspect of the present invention, the subcutaneous preparations can have viscosities (η) ranging from about 1 mPa*s (at 25° C.) to about 2500 mPa*s (at 25° C.), more preferably from about 1 mPa*s (at 25° C.) to about 1000 mPa*s (at 25° C.), and even more preferably from about 1 mPa*s (at 25° C.) to about 500 mPa*s (at 25° C.) as determined using methods described by Anseth, K. S., et al. [Biomaterials, Vol. 17(17): pp. 1647-1657 (1996)], including the use of a cone and plate viscometer. Such ranges are meant to include viscosities ranging from about 1 mPa*s (at 25C) to about 2500 mPa*s (at 25° C.), and values falling within this range, such as viscosities of about 5 mPa*s, about 10 mPa*s, about 25 mPa*s, about 50 mPa*s, about 75 mPa*s, about 100 mPa*s, about 150 mPa*s, about 200 mPa*s, about 250 mPa*s, about 300 mPa*s, about 350 mPa*s, about 400 mPa*s, and about 450 mPa*s, as well as values between any two of these values (e.g., between about 5 mPa*s and about 160 mPa*s).

In accordance with this aspect of the present invention, the invention also refers to the preparation of a medicament for the subcutaneous treatment of chronic wounds. Such subcutaneous pharmaceutical preparations can be carried out according to conventional procedures, for example, using aqueous or substantially aqueous solutions of the active substance or substances are produced, and are then optionally treated with one or more diffusion-inhibiting substances, such that substantially no solid particles are present. In further procedural variants, aqueous solutions of an optionally produced diffusion-inhibiting substance or composition is first produced, and this solution is then subsequently added to the therapeutic agents of the present invention. Still further, the therapeutic agents of the present invention may be admixed with one or more additional substances, including additional therapeutic agents, diffusion-inhibiting substances, pharmaceutical Excipients, and the like. By means of such thus-produced pharmaceutical preparations, it may be possible to utilize therapeutic agents, such as etanercept, efalizumab, adalimumab, alefacept, onercept, and the like to treat chronic wounds subcutaneously in a substantially pain-free and/or medically-safe manner.

Accordingly, by means of the preparation of such subcutaneous dermatological formulations for the treatment of chronic wounds in accordance with aspects of the present invention, highly concentrated doses may be possible that are well tolerated in the patient after subcutaneous administration. Further, it is possible to provide the dermatological formulation in accordance with this aspect of the present invention so that it can be given using a multi-dose administration system.

Kits

The present invention also includes pharmaceutical kits useful, for example, in the treatment or prevention of osteoarthritis or rheumatoid arthritis, which comprise one or more containers containing a pharmaceutical composition comprising a therapeutically effective amount of one or more of the first therapeutic agents. Such kits may further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, may also be included in the kit.

Article of Manufacture

The present invention also encompasses an article of manufacture. As used herein, article of manufacture is intended to include, but not be limited to, kits and packages. The article of manufacture of the present invention, comprises: (a) a first container; (b) a pharmaceutical composition located within the first container, wherein the composition, comprises: a first therapeutic agent, comprising: a compound of the present invention or a pharmaceutically acceptable salt form thereof; and, (c) a package insert stating that the pharmaceutical composition can be used for the treatment of an inflammatory disorder (as defined previously). In another embodiment, the package insert states that the pharmaceutical composition can be used in combination (as defined previously) with a second therapeutic agent to treat an inflammatory disorder. The article of manufacture can further comprise: (d) a second container, wherein components (a) and (b) are located within the second container and component (c) is located within or outside of the second container. Located within the first and second containers means that the respective container holds the item within its boundaries.

The first container is a receptacle used to hold a pharmaceutical composition. This container can be for manufacturing, storing, shipping, and/or individual/bulk selling. First container is intended to cover a bottle, jar, vial, flask, syringe, tube (e.g., for a cream preparation), or any other container used to manufacture, hold, store, or distribute a pharmaceutical product.

The second container is one used to hold the first container and, optionally, the package insert. Examples of the second container include, but are not limited to, boxes (e.g., cardboard or plastic), crates, cartons, bags (e.g., paper or plastic bags), pouches, and sacks. The package insert can be physically attached to the outside of the first container via tape, glue, staple, or another method of attachment, or it can rest inside the second container without any physical means of attachment to the first container. Alternatively, the package insert is located on the outside of the second container. When located on the outside of the second container, it is preferable that the package insert is physically attached via tape, glue, staple, or another method of attachment. Alternatively, it can be adjacent to or touching the outside of the second container without being physically attached.

The package insert is a label, tag, marker, etc. that recites information relating to the pharmaceutical composition located within the first container. The information recited will usually be determined by the regulatory agency governing the area in which the article of manufacture is to be sold (e.g., the United States Food and Drug Administration). Preferably, the package insert specifically recites the indications for which the pharmaceutical composition has been approved. The package insert may be made of any material on which a person can read information contained therein or thereon. Preferably, the package insert is a printable material (e.g., paper, plastic, cardboard, foil, adhesive-backed paper or plastic, etc.) on which the desired information has been formed (e.g., printed or applied).

The following prophetic examples are included to demonstrate preferred embodiments of the invention. It should be appreciated by those of skill in the art that the techniques disclosed in the prophetic examples which follow represent techniques believed by the inventor to function well in the practice of the invention, and thus may be considered to constitute preferred modes for its practice. However, those of skill in the art should, in light of the present disclosure, appreciate that many changes can be made in the specific embodiments which are disclosed and still obtain a like or similar result without departing from the scope of the invention.

### EXAMPLES

### Example 1

Dapsone is added to triacetin in a glass beaker. The resulting mixture is heated to an elevated temperature (above room temperature) with stirring until all of the drug is dissolved. Colloidal silicon dioxide is added to the solution and mixed with a spatula until wetted. The mixture is then sheared on a high speed propeller mixer until a homogeneous gel is formed. The gel can contain about 0.25 wt. % dapsone, about 9.0 wt. % colloidal silicon dioxide, and about 90 wt. % triacetin.

The topical gel as prepared above can then be applied topically, using the appropriate means, to a chronic wound on the surface of a human or animal patient.

### Example 2

Propylene glycol (20.0 g), triacetin (343.0 g) and colchicine (1.0 g) is placed in a glass beaker then heated with stirring until all of the drug is dissolved. Colloidal silicon dioxide (36.0 g) is added to the solution and mixed with a spatula until wetted. The mixture is then sheared on a high speed propeller mixer until a homogeneous gel is formed. The gel contains about 1 wt. % colchicine, 5.0% propylene glycol, 9.0% colloidal silicon dioxide, and 85.75% triacetin.

The topical gel as prepared above can then be applied topically, using the appropriate means, to a chronic wound on the surface of a human or animal patient.

### Example 3

### Preparation of an Etanercept-Containing Hydrogel

Preparation Method 1. 20 mg to 100 mg of the active substance, etanercept, is weighed into a closable glass vessel together with about 5 g of sodium carboxymethylcellulose (Na CMC) and made up to 100 g with distilled water. The solid components is then dispersed in the closed vessel by shaking vigorously for a period of time. Subsequently, the mixture is stirred, initially rapidly, for example at about 300 revolutions per minute, using a magnetic stirrer with a large magnetic core or using a propeller stirrer. After about 10 minutes the number of revolutions is lowered to about 10 to 20 per minute in order to avoid the inclusion of air in the swelling preparation. The temperature can be increased up to about 80° C. in order to accelerate the swelling procedure. The preparation is stirred in the closed vessel until the transparent gel is homogeneous upon flowing and appears free from agglomerations, which may be several days.

Preparation Method 2. 20 mg or 100 mg of the active substance (etanercept) is made up to 95.0 g with bidistilled water and dissolved. About 5 g of Na CMC is weighed into a closable glass vessel and treated with the active substance solution. The solid components is dispersed in the closed vessel by vigorous shaking for 30 seconds. Subsequently, the mixture is stirred, initially rapidly, for example at about 300 revolutions per minute, using a magnetic stirrer with a large magnetic core or using a propeller stirrer. After about 10 minutes the number of revolutions is lowered to about 10 to 20 per minute in order to avoid the inclusion of air in the swelling preparation. The temperature can be increased up to about 80° C. in order to accelerate the swelling procedure. The preparation is stirred in the closed vessel until the transparent gel is homogeneous upon flowing and appears free from agglomerations.

Preparation Method 3. 20 mg or 100 mg of the active substance (etanercept) is weighed into a closable glass vessel together with about 2.5 g of alginate and 100 mg of calcium chloride and is made up to 100 g with bidistilled water. The solid components are dispersed in the closed vessel by shaking vigorously for a period of time. Subsequently, the mixture is stirred, initially rapidly, for example at about 300 revolutions per minute, using a magnetic stirrer with a large magnetic core or using a propeller stirrer. After about 10 minutes the number of revolutions is lowered to about 10 to 20 per minute in order to avoid the inclusion of air in the swelling preparation. The temperature can be increased up to about 80° C. in order to accelerate the swelling procedure. The preparation is stirred in the closed vessel until the transparent gel is homogeneous upon flowing and appears free from agglomerations.

The gel prepared according to method 1, 2, or 3 is then filled into sterilized glass syringes having a flask and rubber plunger, with the fill amount of each syringe being about 0.55 g (filling via the cone). The cone of each syringe is then sealed with a rubber tip cap.

The filled syringes is stood upright (i.e. with the cone upwards) and sterilized by steam scrubbing in an autoclave. A heat conductor placed in the product solution of one syringe can be used to control the sterilization time. The syringes are subsequently dried in a sterile room for 12 hours at room temperature under laminar flow and stored sealed in sterilized foil until used.

Application. The hydrogel formulations can be tested on human or animal patients suffering from one or more chronic wounds, and evaluated histophathologically, clinically, and macroscopically.

### Example 4

### Preparing a Hydrogel-Based Active Substance Carrier

a) Preparing a uni-laminate: polyvinyl pyrrolidone (about 3 wt. %) is dissolved in purified water (about 34 wt. %.), and glycerine (about 27 wt. %) as well as gelatine (about 15 wt. %) are added. The solution is allowed to swell for 10 min., and is thereafter heated to an elevated temperature until the solution is substantially clear. To this solution is added saccharose (about 10 wt. %) and dexpanthenol (about 10 wt. %), one after the other, while stirring continuously. The homogenized solution is subsequently applied to a suitable film or filled in suitable containers (e.g. thermoforming sheets) and is left to set.

b) Preparation of a multi-laminate: carboxymethyl cellulose (CMC) is dispersed in an organic solution consisting of polyvinyl pyrrolidone, PEG 400 and ethanol. This mixture is applied in a defined layer thickness to a siliconized polyester film by means of an applicator unit and dried at about 60° C. for about 30 minutes. The dried layer is now referred to as the polymer-containing layer. Following the drying process, a polyester woven fabric is laminated onto the surfaces of the polymer-containing layer, thus producing a “bi-layer”. For preparation of a multi-laminate, the siliconized polyester film is removed and the upper side of the polymer-containing layer of one “bi-layer” is laminated onto the lower side of the woven polymer fabric of a second “bi-layer”. The result is a wound dressing consisting of four layers.

Separation of Partial Amounts.

If the hydrogel is applied sheet-like to a film, individual punched pieces of hydrogel are subsequently punched out employing a suitable punch, and these can be conveyed to suitable containers (e.g. thermosetting sheets).

Sterilization Method.

Sterilization of the rigid hydrogel is performed by means of ionised rays (radiation sterilization). Preferably, gamma rays are used as these have a great depth of penetration and interact only slightly with the material to be sterilized. The dose to be applied (e.g. 25 kGy) depends on the microbiological starting conditions (F=n×D). As a radiation source, the radionuclide ⁶⁰Co (or a similar radionuclide) is typically used.

Equipping the Active Substance Carrier with Active Substance.

The active substance (e.g. entanercept, adalimumab, or other of the biological agents described herein) is dissolved in sterilized water and is dosed to the sterilised hydrogel, so that the active substance solution evenly wets the surface of the active substance carrier and is absorbed thereby.

Final Packaging.

The hydrogels equipped with active substance carrier and active substance solution is then subsequently packaged, so that sufficient protection from microbiological recontamination is guaranteed.

All of the topical formulations described above, and suggested by the present disclosure, can be tested for their ability to inhibit cytokines such as TNF-α or their related enzymes, such as TACE, inhibit neutrophils, or both, using test methods known in the art.

The invention has been described in the context of preferred and other embodiments and not every embodiment of the invention has been described. Obvious modifications and alterations to the described embodiments are available to those of ordinary skill in the art. The disclosed and undisclosed embodiments are not intended to limit or restrict the scope or applicability of the invention conceived of by the Applicant, but rather, in conformity with the patent laws, Applicant intends to protect all such modifications and improvements to the full extent that such falls within the scope or range of equivalent of the following claims.

What is claimed is:

1. A method of healing a chronic wound in a patient in need thereof, the method comprising the step of topically administering to the patient a dermatological composition comprising a therapeutically effective amount of a TNF antagonist, a neutrophil antagonist, a combination TNF antagonist/neutrophil antagonist, or a combination thereof.

2. The method of healing of claim 1, wherein the chronic wound is selected from the group consisting of venous stasis ulcers, diabetic ulcers, pressure ulcers, vasculitis-induced ulcers, arterial ulcers, and pyoderma gangrenosum.

3. The method of claim 1, wherein the TNF antagonist is selected from the group consisting of adalimumab, alefacept, efalizumab, etanercept, and onercept.

4. The method of claim 1, wherein the neutrophil antagonist is selected from the group consisting of dapsone, colchicine, colchicine derivatives, prodrugs thereof, and polymorphs thereof.

5. The method of claim 1, wherein the combination TNF antagonist/neutrophil antagonist is selected from the group consisting of sulfasalazine, sulfapyridine, mesalamine, and combinations thereof.

6. The method of claim 1, wherein the dermatological composition is a gel, a semi-solid gel, a cream, a lotion, an ointment, a spray, a dispersion, or a salve.

7. The method of claim 1, wherein the therapeutically effective amount is in the range from about 0.01 mg/kg to about 100 mg/kg.

8. The method of claim 1, wherein the dermatological composition further comprises an anti-bacterial agent, an anti-fungal agent, an anti-mycobacterial agent, an anti-inflammatory agent, or a combination thereof.

9. A method for inhibiting the action of TNF for treating chronic wounds in a human, the method comprising:
topically administering a dermatological composition comprising a therapeutically-effective dosage level of a TNF antagonist selected from the group consisting of adalimumab, alefacept, efalizumab, etanercept, and onercept.

10. The method of claim 8, wherein the therapeutically effective dosage level is in the range from about 0.01 mg/kg to about 100 mg/kg.

11. The method of claim 8, wherein the dermatological composition is a gel, a semi-solid gel, a cream, a lotion, an ointment, a spray, a dispersion, or a salve.

12. A method for inhibiting the action of neutrophils for treating chronic wounds in a human, the method comprising:
topically administering a dermatological composition comprising a therapeutically-effective dosage level of a neutrophil antagonist selected from the group consisting of dapsone, colchicine, colchicine analogs, colchicine prodrugs, and colchicine polymorphs.

13. The method of claim 8, wherein the therapeutically effective dosage level is in the range from about 0.01 mg/kg to about 100 mg/kg.

14. The method of claim 8, wherein the dermatological composition is a gel, a semi-solid gel, a cream, a lotion, an ointment, a spray, a dispersion, or a salve.

15. The method of claim 8 or claim 11, wherein the dermatological composition is in the form of a subcutaneous formulation suitable for application topically.

16. A wound dressing having a layered structure for the controlled release of active substance to chronic wounds, the dressing comprising:
a hydrocolloid-containing swellable hydrogel; and
at least one active substance in at least one of the layers,
wherein the at least one active substance is a biologically active peptide or protein, a TNF antagonist, a TNF-alpha antagonist, a neutrophil antagonist, or a combination thereof.

17. The wound dressing of claim 16 wherein the biologically active peptide or protein is adalimumab, alefacept, efalizumab, etanercept, onercept, or a combination of two or more of said biologically active peptides or proteins.

18. A method of healing a chronic wound in a patient in need thereof, the method comprising the step of subcutaneously administering to the patient a dermatological composition comprising a therapeutically effective amount of a TNF antagonist, a neutrophil antagonist, a combination TNF antagonist/neutrophil antagonist, or a combination thereof in a therapeutically effective amount.

19. The method of healing of claim 18, wherein the chronic wound is selected from the group consisting of venous stasis ulcers, diabetic ulcers, pressure ulcers, vasculitis-induced ulcers, arterial ulcers, and pyoderma gangrenosum.

20. The method of claim 18, wherein the dermatological composition is a hydrogel having a viscosity from about 1 mPa*s to about 2500 mPa*s.
